(19) United States
(12) Patent Application Publication
(10) Pub. No.: US 2023/0253125 A1
(43) Pub. Date: Aug. 10, 2023

(54) SYSTEM FOR FACILITATING COMMUNICATION, COLLABORATION, AND QUALITY ASSURANCE FOR THE HOME HEALTHCARE INDUSTRY

(71) Applicant: Latasha Hawk, Ocala, FL (US)

(72) Inventor: Latasha Hawk, Ocala, FL (US)

(21) Appl. No.: 18/082,139

(22) Filed: Dec. 15, 2022

Related U.S. Application Data
(60) Provisional application No. 63/256,296, filed on Oct. 15, 2021.

(51) Int. Cl. G16H 80/00; G16H 10/20; G16H 40/20

(52) U.S. Cl. CPC G16H 80/00; G16H 10/20; G16H 40/20

(57) ABSTRACT

A system for facilitating communication, collaboration, and OASIS quality assurance in the home healthcare industry is disclosed, including at least one user computing device in operable connection with a user network. An application server is in operable communication with the user network to host an application system for providing a platform for facilitating communication, collaboration, and OASIS quality assurance in the home healthcare industry. The application system includes a user interface module for providing access to the application system through the user computing device. The platform provides a means for submitting a collaborative report viewable by the users and a third party.

### CROSS-REFERENCE TO RELATED APPLICATIONS

The present application claims priority to U.S. Provisional Patent Application No. 63/256,296 filed Oct. 15 ,2021, entitled “SYSTEM FOR FACILITATING COMMUNICATION, COLLABORATION, AND QUALITY ASSURANCE FOR THE HOME HEALTHCARE INDUSTRY” which is hereby incorporated by reference in its entirety.

### TECHNICAL FIELD

The embodiments generally relate to computerized systems for aiding the operations for the healthcare industry and more particularly to systems for facilitating communication, collaboration, and quality assurance in the home healthcare industry.

### BACKGROUND

Home healthcare includes various healthcare services provided in the patient's home by licensed medical staff including physical, speech, occupational therapists and assistants, nurses and nurses' assistants, medical social workers, and home health aides, and may comprise a range of activities. Licensed medical staff will hereafter be referred to as clinicians. Home healthcare includes various types of assistance in daily living for ill, disabled, or elderly people who do not need to, or do not want to, be in a clinic or nursing home. Benefits include lower costs while receiving equal or better care when compared to in-clinic settings. Once ordered by a physician, nurses, occupational, physical and/or speech therapist are required to complete a discipline neutral assessment of a patient's needs to determine their eligibility for home healthcare and design a plan of care. Medicare requires the completion of the Outcome and Assessment Information Set (OASIS) for each new patient admitted to home healthcare, as well as other specific time points during the patient's episode of care. The OASIS assessment is tool to collect patient specific data to ensure home health agencies are providing the same standard of quality care. The Centers for Medicare and Medicaid Services uses this tool for Medicare certified home healthcare agencies (HHAs) across the United States. The information gathered is used to determine per patient reimbursement, quality of care ratings, and incentive pay, therefore, accuracy is vital. Currently, clinicians rely on printed material, in-service trainings, generic guidance in the electronic medical records and post assessment feedback from reviewers to ensure accuracy. Due to the isolating nature of home healthcare collaboration and communication regarding patient's care among care providers is expected within the first five days of care. Currently, per patient communication and collaboration among field clinicians and office management occurs via email, telephone, in-person meetings or review of patient medical records. The current system for quality assurance does not rise to meet the expectations set out by Medicare. Additionally, the OASIS assessment is designed to be discipline neutral and standardized, unfortunately, this methodology is not employed by each clinician during every visit leading to inaccurate assessments.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

The embodiments provided herein relate to a system that allows point of service OASIS guidance and a centralized digital platform for collaboration and communication among field clinicians and office management staff. The system will be utilized, in part, as a collaborative assessment tool for onboarding a patient for care to a Home Healthcare Agency (HHA). The system is standalone from electronic medical records systems (EMR) with future iterations to interface with EMR systems to further streamline processes. Current EMR systems only allows one clinician to answer the OASIS functional assessment questions. Clinical managers must review all individual nurse, physical, occupational, and speech therapy evaluations to infer if the initial clinician answered the question correctly. The system allows each clinician that visits the patient within the five-day collaboration window has an opportunity to answer the functional assessment questions. The system facilitates accuracy by creating an assessment structure that incorporates and organizes the nuisances of OASIS guidance through conditional statements to eliminate inaccurate answers. The system aids in improving patient care by neutralizing discipline bias. An efficient process for ensuring an accurate patient assessment will save time, money, and increase efficiency of the homecare processes.

In some aspects, the system facilitates the generation of a collaborative report. The collaborative report is generated by each clinician completing Oasis questions within the collaboration period. The report provides a quick overview of the patient's performance over the assessment period by detailing each clinicians' OASIS functional score answers, as well as additional patient information such as, however not limited to, treatment plans, treatment frequency, identification of new problems, caregiver support, priority of care, or any pertinent information to improve patient care. The report serves as documentation of collaboration among clinicians and can be downloaded to the patient's medical record. The clinical manager or OASIS reviewer can use the report to ensure that the final OASIS assessment that is submitted to Medicare is an accurate reflection of the patient's functional performance at the time of admission to home healthcare. Current systems lack a single patient specific collaborative report to document multiple OASIS functional score assessment and pertinent patient notes. The report streamlines collaboration and communication at all OASIS key time points.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of a computing system, according to some embodiments;

FIG. 2 illustrates a block diagram of the application program and computing system, according to some embodiments;

FIG. 3 illustrates a screenshot of an exemplary graphical user interface, according to some embodiments;

FIG. 4 illustrates a screenshot of an exemplary graphical user interface, according to some embodiments;

FIG. 5 illustrates a screenshot of an exemplary graphical user interface, according to some embodiments;

FIG. 6 illustrates a screenshot of an exemplary graphical user interface, according to some embodiments;

FIG. 7 illustrates a screenshot of an exemplary graphical user interface, according to some embodiments; and

FIG. 8 illustrates a screenshot of an exemplary graphical user interface, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before describing in detail exemplary embodiments, it is noted that the embodiments reside primarily in combinations of components and procedures related to the system. Accordingly, the system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, and/or computer program product at any possible technical detail level of integration. A computer program product can include, among other things, a computer-readable storage medium having computer-readable program instructions thereon for causing a processor to carry out aspects of the present disclosure.

The embodiments described herein relate to a computerized system for facilitating communication, collaboration, and quality assurance in the home healthcare industry. The system allows clinicians to log in to the platform at the patient's home and complete assessments at the point of care. The system may provide standardized guidance to ensure the accuracy of the assessment of the patient's characteristics. A calendar feature allows clinicians to collaborate on treatment frequency. A notification system allows clinicians and clinical managers to collaborate and communicate with one another in a centralized platform.

In some embodiments, the system facilitates clinical manager's collaborative review of the patient's care prior to finalizing the plan of care.

In some embodiments, the clinician may be alerted to the number of days remaining to complete an assessment before Medicare's window of collaboration closes.

The standalone system allows for the separation of key patient information that impacts financial reimbursement from and voluminous clinical patient information within an electronic medical record.

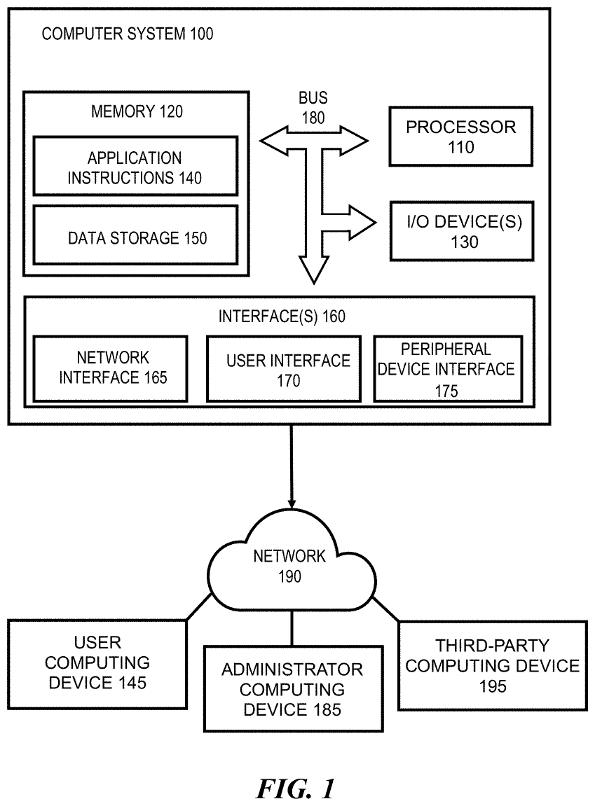

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantum-dot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user's computer, partly on the user's computer, as a stand-alone software package, partly on the user's computer and partly on a remote computer, or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user's computer.

In some embodiments, the computer system 100 may include a caregiver computing device 145, an administrator computing device 185 and a third-party computing device 195 each in communication via the network 190.

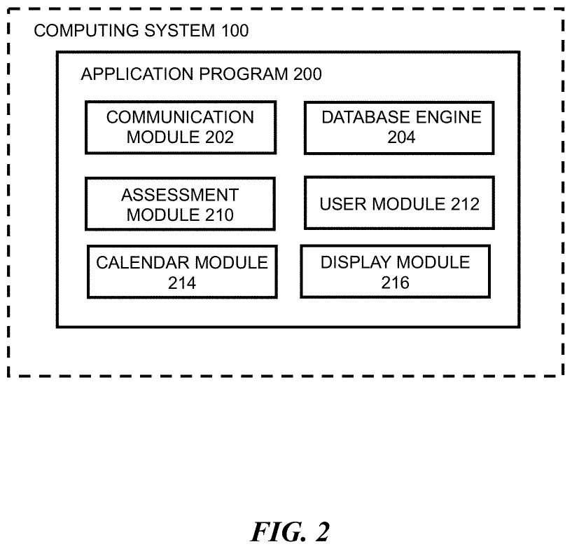

FIGS. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, an assessment module 210, a user module 212, a calendar module 214, a display module 216.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195. In some embodiments, the communication module 202 allows each user to transmit and receive information which may be used by the system. A notification system allows clinicians and clinical managers to collaborate and communicate with one another in a centralized platform.

In some embodiments, the communication module 202 is in operable communication with the system to communicate positive reinforcement and/or to allow users to communicate with one another in support groups. The communication module 202 may also communicate new aversion therapy plans developed by the system.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations. The database engine 204 allows each user and module associated with the system to transmit and receive information stored in various databases.

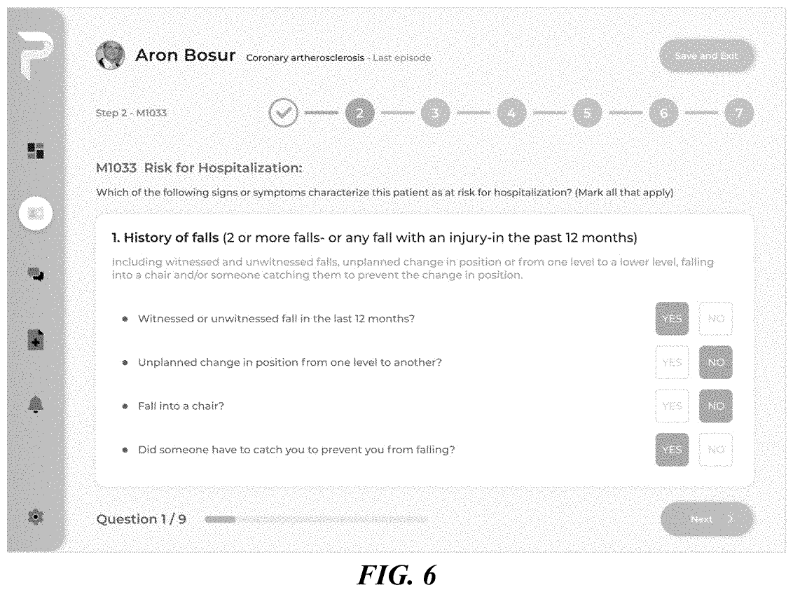

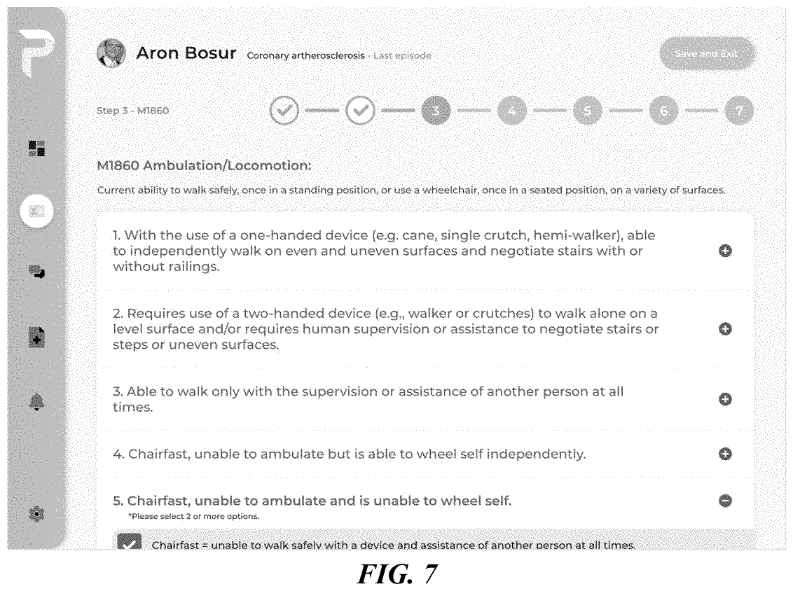

In some embodiments, the assessment module 210 is configured to generate and transmit, via the communication module 202 a questionnaire of a patient. The assessment module 210 may then receive the answers to the questionnaire which have been input by the user to allow the system to make an assessment and provide standardized guidance to ensure the accuracy of the assessment of the patient's characteristics. An exemplary patient questionnaire is provided in FIGS. 6-7.

In some embodiments, the user module 212 facilitates the creation of a user account for the application system. The user module 212 may allow the user to input account information, establish user permissions and the like. The user module 212 may be in communication with the display module to illustrate user information, patient schedules, messages, and other information associated with patient care.

In some embodiments, the calendar module 214 is configured to allow clinicians to collaborate on treatment frequency. The calendar module 214 may provide, via a display module 216 a calendar interface showing the treatment frequency, who performed the treatment or otherwise interacted with the patient.

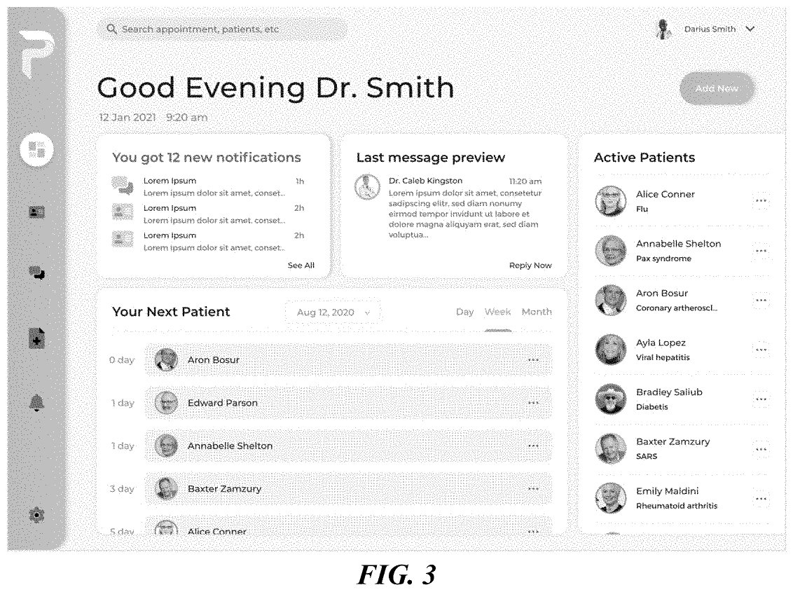

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored. Further, the display module is operable to display the exemplary screenshots illustrated and described in FIGS. 3-8 below.

FIGS. 3-8 illustrates screenshots of the user interface which allows caregivers to collaborate, communicate, and perform quality assurance tasks in the homecare setting. The system will be utilized, in part, as a collaborative assessment tool to assist in onboarding a patient to the Home Health Agency (HHA) and patient management system (EMR). The platform is beneficial during key OASIS time points including Start of Care, Resumption of Care, Follow-up, Significant Change in Conditions and Discharge time points. The user interface allows patient profiles to be created while allowing caregivers to indicate patient priorities, goals of treatment, and input patient notes, which can be shared with other clinicians' who may interact with the patient.

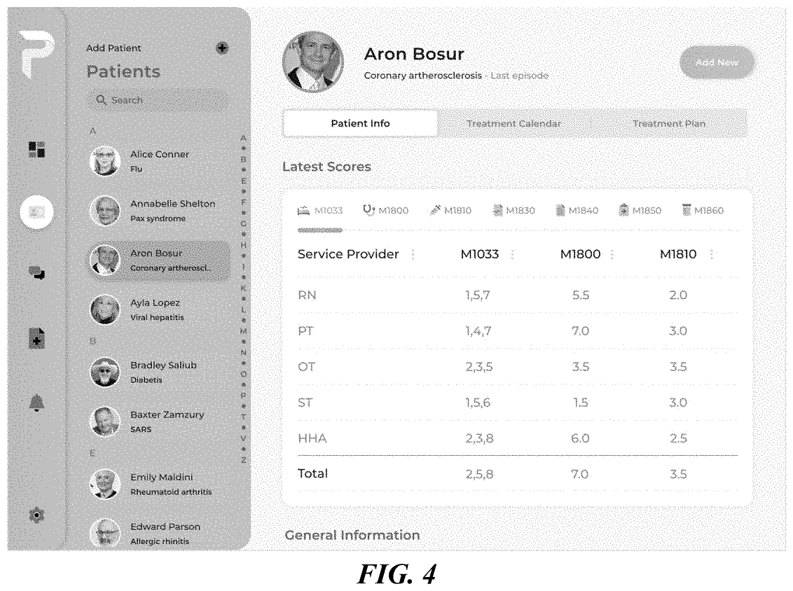

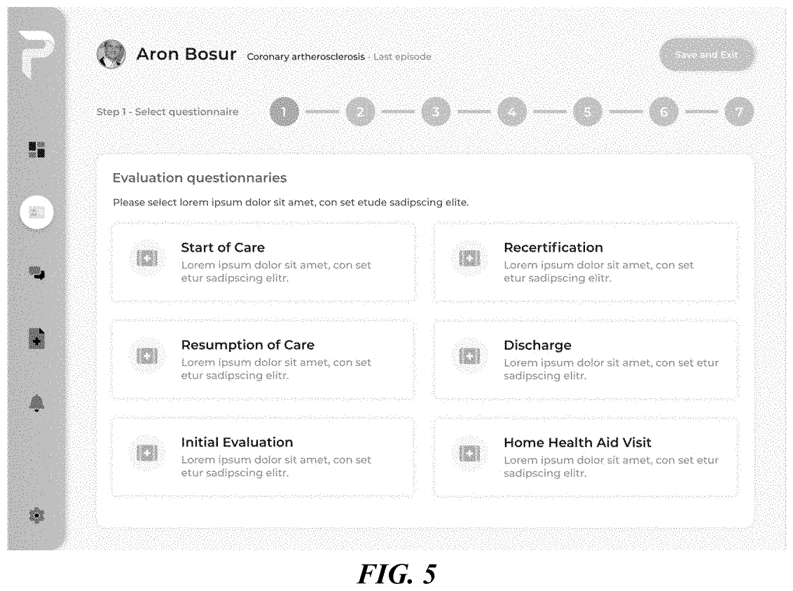

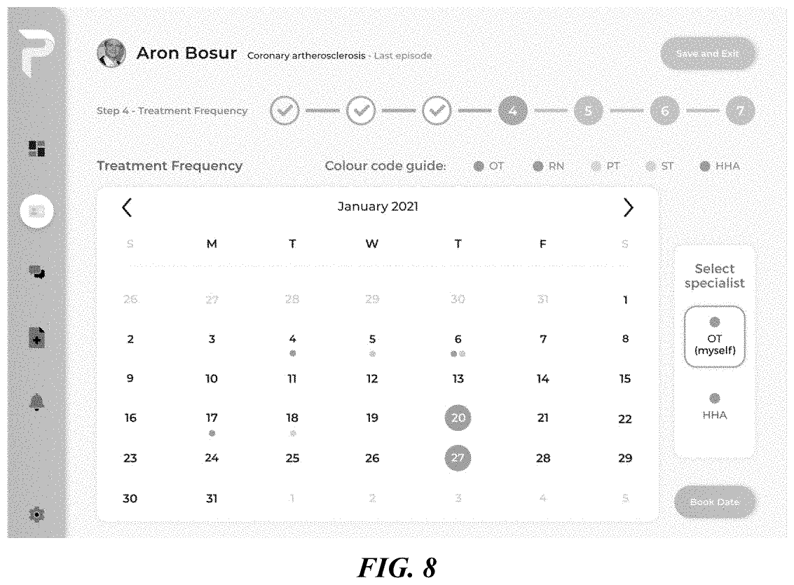

In particular, FIG. 3 illustrates the user interface wherein the clinician may view notifications/messages, and a patient list. The numbered days on the left side of the patient's name identifies the number of days remaining in the collaborative window. FIG. 4 illustrates an example of a collaborative report. FIG. 5 illustrates the interface of the OASIS time points for which a clinician can choose to initiate an assessment. This may include start of care, resumption of care, recertification (follow-up), discharge, and home health aide visit information. FIGS. 6 and 7 illustrate the questionnaire interface to show an exemplary OASIS questionnaire with embedded guidance. FIG. 8 illustrates the calendar interface.

In some embodiments, a collaborative report may be generated using the system. The collaborative report allows the clinicians and/or clinical manager to have a centralized place to review all assessments results to compare accuracy of answers over the collaboration window. The collaborative report may be generated from answers to questions provided on a patient questionnaire. The collaborative report includes questions which are required by Medicare to assess the patient's condition to determine a plan of care and to determine how the HHA will be paid for services.

In some embodiments, the system includes the use of various tools to provide clinical managers the ability to analyze therapy assessment guidance. This may allow clinical managers to provide clinical advice.

In some embodiments, a treatment frequency calendar is provided which allows caregivers to collaboratively plan and schedule treatments over a 60-day period of care. The calendar provides various means to ensure that patient care is spread over the episode.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer's job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for facilitating communication, collaboration, and quality assurance in the home healthcare industry, the system comprising:
at least one user computing device in operable connection with a user network;
an application server in operable communication with the user network, the application server configured to host an application system for providing a platform for facilitating communication, collaboration, and quality assurance in the home healthcare industry, the application system having a user interface module for providing access to the application system through the user computing device, the platform for providing a means for submitting a collaborative report viewable by the users and a third party.

2. The system of claim 1, further comprising a communication module to facilitate the communication between licensed field medical staff and office clinical managerial staff

3. The system of claim 2, further comprising an assessment module to transmit, via the communication module, a questionnaire to other clinical home healthcare staff.

4. The system of claim 3, wherein the assessment module receives one or more responses to the questionnaire submitted by clinicians.

5. The system of claim 4, wherein the assessment module provides point of service nuanced and standardized OASIS guidance to improve accuracy.

6. The system of claim 1, further comprising a calendar module to provide a scheduling system for patient care.

7. The system of claim 6, wherein the calendar module allows one or more clinicians to collaborate on treatment frequency.

8. A system for facilitating communication, collaboration, and quality assurance in the home healthcare industry, the system comprising:
at least one user computing device in operable connection with a user network;
an application server in operable communication with the user network, the application server configured to host an application system for providing a platform for facilitating communication, collaboration, and quality assurance in the home healthcare industry, the application system having a user interface module for providing access to the application system through the user computing device, the platform for providing a means for submitting a collaborative report viewable by the users and a third party;
an assessment module to document clinicians' assessment of a patient.

9. The system of claim 8, further comprising a communication module to facilitate the communication and collaboration of pertinent patient information between and office medical staff.

10. The system of claim 9, further comprising an assessment module to transmit, via the communication module, a collaborative report which can be downloaded to the medical record.

11. The system of claim 10, wherein the assessment module identifies days remaining in Medicare's collaborative window.

12. The system of claim 11, wherein the assessment module provides a centralized platform for each clinician to answer OASIS questions that impact financial returns to the HHA prior to submission of plan of care.

13. The system of claim 12, further comprising a notification/messaging module to provide a push notifications clinicians assigned to a patient.

14. The system of claim 13, wherein the calendar module allows one or more clinicians to collaborate on treatment frequency.

15. The system of claim 8, further comprising a calendar interface to display a patient care frequency.

16. The system of claim 8, further comprising a questionnaire interface provided by the assessment module.

17. The system of claim 8, wherein one or more tools provide clinical managers the ability to analyze therapy assessment guidance.
